SEQUENCE LISTING (6 sequences)

SEQ ID NO: 1 - RNA, 21 nt, Artificial
  siRNA
aacaucugccuagaucggcua

SEQ ID NO: 2 - RNA, 19 nt, Artificial
  siRNA
uagcggaucuaggcagaug

SEQ ID NO: 3 - DNA, 18 bp, Artificial
  Primer
gccagagagccaggagca

SEQ ID NO: 4 - DNA, 28 bp, Artificial
  Primer
acacagataaacttggtcttcaggtatg

SEQ ID NO: 5 - DNA, 21 bp, Artificial
  Primer
ttctacaatgagctgcgtgtg

SEQ ID NO: 6 - DNA, 20 bp, Artificial
  Primer
ggggtgttgaaggtctcaaa

(19) United States
(12) Patent Application Publication
(10) Pub. No.: US 2009/0264483 A1
(43) Pub. Date: Oct. 22, 2009

(54) METHODS OF USING PPAR-gamma AGONISTS AND CASPASE-DEPENDENT CHEMOTHERAPEUTIC AGENTS FOR THE TREATMENT OF CANCER

(75) Inventor: John S. Yu, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/373,823

(22) PCT Filed: Jul. 10, 2007
(86) PCT No.: PCT/US07/73138

§ 371 Date: Jan. 14, 2009

Related U.S. Application Data
(60) Provisional application No. 60/807,386, filed on Jul. 14, 2006.

(51) Int. Cl. A61K 31/427; A61K 31/357; A61K 31/337

(52) U.S. Cl. 514/369; 514/452; 514/449

(57) ABSTRACT

This invention relates to compositions and methods utilizing a caspase-dependent chemotherapeutic drug and a PPARy-agonist, such as VP-16 or Taxol as the chemotherapeutic drug and troglitazone or pioglitazone as the PPARy agonist, for the treatment of cancer, including glioblastoma multiforme. The present invention demonstrates that PPAR agonists work with caspase-dependent chemotherapeutic drugs to increase the cytotoxic effects of these drugs in glioma cells.

### FIELD OF INVENTION

This invention relates to compositions and methods for the treatment of cancer in a mammal in need thereof.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Cancer remains among the leading causes of death in the United States and around the world. Various forms of cancer are differentially treated, depending in part on the location of a tumor. One particularly difficult group of tumors to treat includes those that reside in and near the brain. Treatment of brain tumors presents a number of problems, not the least of which being the dangers inherent in any surgical procedure involving regions of the brain and the tissue located nearby. There is little room for error and the consequences of even a minor surgical mishap can be devastating to a patient; brain damage, or even death may result. Still, where possible, surgery remains the preferred method of treatment for most brain tumors and is often performed in conjunction with radiation therapy and chemotherapy. However, even commonly referenced medical authority suggests that patients with brain tumors be referred to centers specializing in investigative therapies; an indication that conventional modes of treatment are not overwhelmingly successful.

Glioblastoma multiforme and anaplastic astrocytomas are classified in the category of brain tumors commonly known as malignant gliomas. Although not particularly common tumors themselves, they represent a class of tumors associated with significant rates of mortality and morbidity. Indeed, brain tumors are the third-most frequent cause of cancer-related death in middle-aged males and the leading cause of cancer death in children. According to the National Brain Tumor Foundation, approximately 190,000 people are diagnosed with primary or metastatic brain tumors in the United States each year. According to the Society for Neuroscience, approximately 20,000 cases of glioma are diagnosed each year, and more than half die within 18 months. For patients with the most severe, aggressive form of glioblastoma multiforme (“GBM”), median survival is less than a year. Current treatment for malignant glioma consists of surgical resection followed by radiation therapy and chemotherapy. However, this treatment generally fails in substantially changing the outcome for a patient. Thus, there remains a significant need in the art for improved methods for the treatment of cancer, and, in particular, for brain tumors.

Malignant gliomas adopt the ability to bypass or disrupt fail-safe mechanisms, such as programmed cell death and host immune defense (Chakravarti et al. (2004) Oncogene 23, 7494-7506; Bobola et al. (2004) Clin. Cancer Res. 10, 7875-7883; Akasaki et al. (2004) J. Immunol. 173, 4352-4359), which make current therapeutic interventions ineffective at eradicating residual tumor reservoirs. Because many adjuvant therapies for malignant tumors seek to induce tumor cell apoptosis, strategies that lower thresholds for the induction of apoptosis, which can then make other treatments more effective, may improve patient outcomes. Apoptosis is a form of programmed cell death, generally occurring in an ordered sequence of steps, where normal apoptosis ends up conferring a benefit and defective apoptosis can lead to uncontrolled cell proliferation (i.e., cancer). Further understanding of the biological anti-apoptotic mechanisms that govern resistance to conventional glioma therapies is required in order to develop safer and more effective treatments.

Caspases, a family of proteases, play a key role in apoptosis. Initiator caspases, (e.g., caspase-8 and -9), activate effector caspases, (e.g., caspase-3 or -7). The effector caspases cleave other protein substrates within the cell, and the result is the apoptotic process. Tumor necrosis factor-related apoptosis-inducing ligand (“TRAIL”) is a promising anti-neoplastic agent because it induces apoptosis in cancer cells with only a negligible effect on normal cells (Ashkenazi et al., (1999) J. Clin. Investig. 104, 155-162; Hao et al. (2001) Cancer Res. 61, 1162-1170.). It has been known that TRAIL triggers the caspase cascade through interaction with TRAIL-responsive death receptor-γ (“DR”), such as DR4 and DR5, which induce cleavage of caspace-8 in the Fas-associated death domain protein dependent mechanism (Thomas et al. (2004) J. Biol. Chem. 279, 32780-32785.). In this pathway, cleaved caspase-8 plays a significant role as an initiator that can process other members in the caspase cascade. Cleaved caspase-8 induces cleavage of Bid, which upregulates mitochondrial cytochrome c (“Cyt-c”) release (Kuwana et al. (1998) J. Biol. Chem. 273, 16589-16594.). Cyt-c then cooperates with apoptotic protease-activating factor-1 to activate caspase-9 (Li et al. (1997) Cell 91, 479-489). After these activations, both caspase-8 and -9 activate caspase-3, which is the primary activator of apoptotic DNA fragmentation and leads to cancer cell apoptosis (Wolf et al. (1999) J. Biol. Chem. 274, 30651-30656.).

Despite the numerous reports describing the favorable anti-tumor activities of TRAIL, malignant gliomas exhibit considerable heterogeneity in their sensitivity to TRAIL, even among those expressing DR4 and DR5 (Hao et al. (2001) Cancer Res. 61, 1162-1170.; Knight et al. (2001) Oncogene 20, 5789-5798.). In this regard, it has been reported recently that several cancers, including gliomas, constitutively express FLIP (Fas-associated death domain-like IL-1α-converting enzyme-inhibitory protein) (Yoon et al. (2002) J. Neurooncol. 60, 135-141; Krueger et al. (2001) Mol. Cell. Biol. 21, 8247-8254; Zhang et al. (2004) Cancer Res. 64, 7086-7091.), which is a cytoplasmic protein that inhibits the recruitment and processing of caspase-8 (Krueger et al. (2001) Mol. Cell. Biol. 21, 8247-8254.), and that the overexpression of FLIP induces cancers resistance to DR-dependent apoptosis (Rippo et al. (2004) Oncogene 23, 7753-7760.). In addition, it has also been found that Bcl-2 family proteins modulate caspase-9 activity by controlling the permeability of mitochondrial membranes and that dysregulation of these proteins in cancer cells correlates with their anti-apoptotic potential and progression (Reed, J. C. (1999) Curr. Opin. Oncol. 11, 68-75.). Specifically, the anti-apoptotic Bcl-2 family, including Bcl-2 and Bcl-xL, stabilizes the mitochondrial porin channel (voltage-dependent anion channel) and inhibits Cyt-c release (Shimizu et al. (1999) Nature 399, 483-487.), whereas the pro-apoptotic Bcl-2 family, including Bax and Bad, antagonizes this process by competitive heterodimerization with anti-apoptotic Bcl-2 proteins (Adams et al. (1998) Science 281, 1322-1326.). Although down-regulation of FLIP and/or anti-apoptotic Bcl-2 family proteins are therefore a therapeutic target for promotion of caspase cascade activity, the mechanism that regulates these proteins in glioma cells is not fully understood.

Peroxisome proliferator-activated receptor-γ (“PPARγ”) is a member of the nuclear hormone receptor superfamily of ligand-activated transcription factors. PPARγ agonists/ligands include thiazolidinediones (“TZD”). TZDs are currently used to treat diabetes, and include troglitazone (“TGZ”), pioglitazone (“PGZ”), rosiglitazone (“RGZ”), and ciglitazone (“CGZ”). It is known that PPARγ agonists exert anti-tumor effects in a variety of cancers, indicating anti-proliferative, anti-angiogenic, and pro-differentiation effects (Froehlich et al., Action of Thiazolidinediones on Differentiation, Proliferation and Apoptosis of Normal and Transformed Thyrocytes in Culture, Endocrine-Related Canc. (June 2005), 12(2), pp. 291-303.; Koeffler, H. P., The Cellular Response of PPARγ Ligands is Related to the Phenotype of Neuroblastoma Cell Lines, Oncol. Res. (2004), 14(7-8), pp. 345-354.; Dong et al., Regulatory Effects of Peroxisome Proliferator-activated Receptor γ on the Growth of Pancreatic Carcinoma, Zhonghua Nei Ke Za Zhi (2003), 42(7), pp. 479-82.). TGZ's effect on various cancer cells, combinations of TGZ with other agents to treat cancer, the effect of PPARγ agonists and ligands for retinoic acid receptor (“RAR”) and retinoid X receptor (“RxR”) on cancer cell treatment, and the effect of combined treatment with TZDs and TRAIL on TRAIL-induced apoptosis of cancer cells are discussed in the art (Froehlich et al., Action of Thiazolidinediones on Differentiation, Proliferation and Apoptosis of Normal and Transformed Thyrocytes in Culture, Endocrine-Related Canc. (June 2005), 12(2), pp. 291-303.; Kubota et al., Ligand for Peroxisome Proliferator-activated Receptor γ (Troglitazone) Has a Potent Antitumor Effect Against Human Prostate Cancer Both in vitro and in vivo, Canc. Res. (1998), 58(15), pp. 3344-3352.; Barrera et al., Synergistic Effect of 4-Hydroxynonenal and PPAR Ligands in Controlling Human Leukemic Cell Growth and Differentiation, Free Radical Bio. and Med. (2002), 32(3), pp. 233-245.; Stoll, B. A., Linkage Between Retinoid and Fatty Acid Receptors: Implications for Breast Cancer Prevention, Eur. J. of Canc. Prev. (2002), 11(4), pp. 319-325.; Elstner et al., Ligands for Peroxisome Proliferator-activated Receptor γ and Retinoic Acid Receptor Inhibit Growth and Induce Apoptosis of Human Breast Cancer Cells in vitro and in BNX Mice, Proc. of the Natl. Acad. of Sci. (1998), 95(15), pp. 8806-8811.; Yao et al., Dramatic Synergistic Anticancer Effect of Clinically Achievable Doses of Lovastatin and Troglitazone, Int. J. Canc. (1998), 118(3), pp. 773-779.; Yang et al., Activation of the PPAR Pathway Induces Apoptosis and COX-2 Inhibition in HT-29 Human Colon Cancer Cells, Carcinogenesis (2001), 22(9), pp. 1379-1383.). PPARγ agonists, including TGZ, RGZ, and PGZ, have been shown to enhance cancer cell apoptosis induced by TRAIL, and to sensitize TRAIL-resistant cells to TRAIL-induced apoptosis (Lu et al., Peroxisome Proliferator-activated Receptor γ Agonists Promote TRAIL-induced Apoptosis by Reducing Survivin Levels via Cyclin D3 Repression and Cell Cycle Arrest, J. of Bio Chem., 280(8), pp. 6742-6751.; Göke et al., Regulation of TRAIL-induced Apoptosis by Transcription Factors, Cell. Immunol. (2000), 201, pp. 77-82.; Göke et al., Pioglitazone Inhibits Growth of Carcinoid Cells and Promotes TRAIL-induced Apoptosis by Induction of p21^{waf1/cip1}, Digestion (2001), 64(2), pp. 75-80.; Park et al., Cotreatment with Pioglitazone, a Synthetic Ligand for Peroxisome Proliferator-activated Receptor γ (PPARγ), Enhances Tumor Necrosis Factor-related Apoptosis-inducing Ligand (TRAIL) Induced Apoptosis of Human Leukemia Cells, Blood (2004), 104(11, Pt. 2), p. 178B.). It is now recognized that many PPARγagonists have pro-apoptotic activities that are induced through PPARγ-independent pathways (Lu et al., Peroxisome Proliferator-activated Receptor γ Agonists Promote TRAIL-induced Apoptosis by Reducing Survivin Levels via Cyclin D3 Repression and Cell Cycle Arrest, J. of Biol. Chem., 280(8), pp. 6742-6751.; Göke et al., Regulation of TRAIL-induced Apoptosis by Transcription Factors, Cell. Immunol. (2000), 201, pp. 77-82.; Göke et al., Pioglitazone Inhibits Growth of Carcinoid Cells and Promotes TRAIL-induced Apoptosis by Induction of p21^{waf1/cip1}, Digestion (2001), 64(2), pp. 75-80.; Park et al., Cotreatment with Pioglitazone, a Synthetic Ligand for Peroxisome Proliferator-activated Receptor γ (PPARγ), Enhances Tumor Necrosis Factor-related Apoptosis-inducing Ligand (TRAIL) Induced Apoptosis of Human Leukemia Cells, Blood (2004), 104(11, Pt. 2), p. 178B.).

Certain chemotherapeutic drugs are caspase-dependent and induce apoptosis of cancer cells, (e.g., VP-16 (generic name, etoposide), Taxol (generic name, paclitaxel)). The cytotoxic activities of these caspase-dependent drugs are based on induction of apoptosis by activation of caspase-2, -8, -9, and -10.

### SUMMARY OF THE INVENTION

The following embodiments and aspects thereof are described and illustrated in conjunction with compositions, methods and kits are meant to be exemplary and illustrative, not limiting in scope.

The present invention describes a method of treating cancer in a mammal in need thereof, comprising: providing a caspase-dependent chemotherapeutic agent and a peroxisome proliferator-activated receptor-γ (“PPARγ”) agonist; and administering a therapeutically effective amount of the caspase-dependent chemotherapeutic agent and a therapeutically effective amount the PPARγ agonist to the mammal in need of treatment for cancer. The method may further comprise identifying a mammal in need of treatment for a cancer that is resistant to induction of apoptosis.

In various embodiments, the caspase-dependent chemotherapeutic agent used in the inventive method may be etoposide (e.g., VP-16), paclitaxel (e.g., Taxol), temozolomide, BCNU, adriamycin, cpt-11, 5-fluorouracil, oxaliplatin, pemetrexed, or gefitinib. In a particular embodiment, the caspase-dependent chemotherapeutic agent may be etoposide. In another particular embodiment, the caspase-dependent chemotherapeutic agent may be paclitaxel.

In various embodiments, the PPARγ agonist may be troglitazone (“TGZ”), pioglitazone (“PGZ”), rosiglitazone (“RGZ”), or ciglitazone (“CGZ”). In one particular embodiment, the PPARγ agonist may be TGZ.

In various embodiments, the cancer treated by the present inventive method may be breast cancer, colon cancer, prostate cancer, pancreatic cancer, cervical cancer, thyroid cancer, or brain cancer. In a particular embodiment, the cancer treated by the present inventive method is malignant glioma. In particular, the malignant glioma may be gliobastoma multiforme or anaplastic astrocytoma. In another particular embodiment, the cancer treated by the present inventive method is resistant to induction of apoptosis.

The present invention also provides for a composition for the treatment of cancer in a mammal in need thereof comprising: a caspase-dependent chemotherapeutic agent; and a peroxisome proliferator-activated receptor-γ (“PPARγ”) agonist. The caspase-dependent chemotherapeutic agent may be etoposide (e.g., VP-16), paclitaxel (e.g., Taxol), temozolomide, BCNU, adriamycin, cpt-11, 5-fluorouracil, oxaliplatin, pemetrexed, or gefitinib. In a particular embodiment, the caspase-dependent chemotherapeutic agent may be etoposide. In another particular embodiment, the caspase-dependent chemotherapeutic agent may be paclitaxel.

In various embodiments the PPARγ agonist used in the composition may be troglitazone (“TGZ”), pioglitazone (“PGZ”), rosiglitazone (“RGZ”), or ciglitazone (“CGZ”). In a particular embodiment, the PPARγ agonist may be TGZ.

The present invention further provides for a kit for the treatment of cancer in a mammal in need thereof, comprising: a quantity of a caspase-dependent chemotherapeutic agent; a quantity of a peroxisome proliferator-activated receptor-γ (“PPARγ”) agonist; and instructions to administer a therapeutically effective amount of the caspase-dependent chemotherapeutic agent and a therapeutically effective amount the PPARγ agonist to the mammal in need of treatment for cancer. The kit may further comprise instructions to identify a mammal in need of treatment for a cancer that is resistant to induction of apoptosis.

In various embodiments, caspase-dependent chemotherapeutic agent in the kit may be etoposide (e.g., VP-16), paclitaxel (e.g., Taxol), temozolomide, BCNU, adriamycin, cpt-11, 5-fluorouracil, oxaliplatin, pemetrexed, or gefitinib. In a particular embodiment, the caspase-dependent chemotherapeutic agent in the kit may be etoposide. In another particular embodiment, the caspase-dependent chemotherapeutic agent in the kit may be paclitaxel.

In various embodiments, the PPARγ agonist in the kit may be troglitazone (“TGZ”), pioglitazone (“PGZ”), rosiglitazone (“RGZ”), or ciglitazone (“CGZ”). In a particular embodiment, the PPARγ agonist in the kit may be TGZ.

In various embodiments, the kit may be configured to treat a cancer selected from the group consisting of breast cancer, colon cancer, prostate cancer, pancreatic cancer, cervical cancer, thyroid cancer, brain cancer and combinations thereof. In a particular embodiment, the kit may be configured to treat malignant glioma. In other particular embodiments, the kit may be configured to treat gliobastoma multiforme or anaplastic astrocytoma. In still other particular embodiment, the kit may be configured to treat a cancer that is resistant to induction of apoptosis.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows DR4/5 expression and TRAIL sensitivity in malignant gliomas in accordance with an embodiment of the present invention. FIG. 1A: expression levels of DR4 and DR5 in LN-18, U-87MG, and MG-328 cells were analyzed by Western blot. FIG. 1B: cells were treated with human rTRAIL (300 ng/ml) and then stained with Ann and propidium iodide (PI) after 2 and 24 h of treatment. Cells were analyzed by FACS. The data presented are the representative results of LN-18. FIG. 1C: cells were treated with human rTRAIL (1 7.5˜300 ng/ml) for 24 h and then stained with Ann and PI. Cells that stained negative for both Ann and PI were defined as viable cells. Data are means+/−S.D. of three independent experiments.

FIG. 2 shows the role of STAT3 in causing resistance to TRAIL in glioma cells in accordance with an embodiment of the present invention. FIGS. 2A and 2B: STAT3-siRNA (50-600 pmol) or nonsilencing siRNA (600 pmol) was transfected to glioma cells (LN-18) with Oligofectamine reagent. Cells that transfected with Oligofectamine alone were referred to as vehicle. Cells were used for experiments 24 h after transfection. FIG. 2A: total RNA samples extracted from cells were subjected to real time quantitative PCR. FIG. 2B: protein samples extracted from cells were subjected to Western blot. FIGS. 2C, 2D and 2E: STAT3-siRNA (600 pmol) or nonsilencing siRNA (600 pmol) was transfected to glioma cells. Cells that transfected with Oligofectamine alone were referred to as vehicle. FIG. 2C: protein samples extracted from cells were subjected to Western blot. FIG. 2D: cells were treated with rTRAIL (100 ng/ml) for 24 h and then were stained with Ann and PI. Several groups were cultured for 24 h without rTRAIL treatment. Cells that stained negative for both Ann and PI were defined as viable cells. Data are mean+/−S.D. of three independent experiments. ** refers to statistical significance (p<0.01). FIG. 2E: the activities of caspase-3, -8, and -9 were measured by enzyme activity assay after 2 h of the TRAIL treatment. Nonsilencing controls were used for control. The specific activities on each sample were calculated according to the manufacturer's protocol. Data are means+/−S.D. of three independent experiments; ** refers to statistical significance (p<0.01) compared with each control.

FIG. 3 shows the effect of TG on TRAIL in accordance with an embodiment of the present invention. FIG. 3A: expression of PPARγ in LN-18, U-87MG, and MG-328 cells was analyzed by Western blot. FIG. 3B: treatment of cells with TG (30 μM) for 24 h was followed by rTRAIL (100 ng/ml) for 24 h. Several groups were cultured for 48 h without rTRAIL treatment. Cells were stained with Ann and PI. Cells that stained negative for both Ann and PI were defined as viable cells. Data are means+/−S.D. of three independent experiments; ** refers to statistical significance (p<0.01). FIG. 3C: protein samples extracted from cells with and without TG treatment were subjected to Western blot.

FIG. 4 shows TG activity via PPARγ-dependent and -independent pathways in accordance with an embodiment of the present invention. FIGS. 4A, 4B and 4C: cells were treated with GW (20 μM) and/or TG (30 μM) for 24 h. FIG. 4A: protein samples extracted from the cells were subjected to Western blot. FIG. 4B: treatment of cells with TG and/or GW was followed by rTRAIL (100 ng/ml) treatment for 24 h. Cells not treated with TG or GW were used for control. Cells were stained with Ann and PI. Cells that stained negative for both Ann and PI were defined as viable cells. Data are means+/−S.D. of three independent experiments; ** refers to statistical significance (p<0.01) compared with each control. FIG. 4C: treatment of cells with TG and/or GW was followed by rTRAIL (100 ng/ml) treatment for 2 h. Cells not treated with TG or GW were used for control. The activities of caspase-3, -8, and -9 were measured by enzyme activity assay. The specific activities on each sample were calculated according to the manufacturer's protocol. Data are means+/−S.D. of three independent experiments; * refers to statistical significance (p<0.05); ** refers to statistical significance (p<0.01) compared with each control.

FIG. 5 shows expression of PTP1B and SHP-1 after treatment with TG in glioma cells in accordance with an embodiment of the present invention. Protein samples extracted at different time points after treatment with TG (30 μM) were subjected to Western blot in which the expressions of pY705-STAT3, PTP1B, and SHP-1 were analyzed.

FIG. 6 shows the significance of PTP1B in the effect of TG in accordance with an embodiment of the present invention. FIG. 6A: cells were treated with TG (30 μM) and/or PTP inhibitor (PTPI, 50 μM) for 24 h. Protein samples extracted from the cells were subjected to Western blot. FIG. 6B: cells were treated with TG (30 μM) and/or SHP-1 inhibitor (SHPI, 200 μM) for 24 h. Protein samples extracted from the cells were subjected to Western blot. FIG. 6C: treatment of cells with TG and/or PTPI was followed by rTRAIL (100 ng/ml) for 2 h. Cells not treated with TG or PTPI were used for control. The activities of caspase-3, -8, and -9 were measured by enzyme activity assay. The specific activities on each sample were calculated according to the manufacturer's protocol. Data are mean+/−S.D. of three independent experiments; ** refers to statistical significance (p<0.01) compared with each control. FIG. 6D: STAT3-siRNA (600 pmol) or nonsilencing siRNA (600 pmol) was transfected to glioma cells. Cells that transfected with Oligofectamine alone were referred to as vehicle. Cells were used for experiments 24 h after transfection. The activities of caspase-3, -8, and -9 were measured similar to FIG. 6C. Data are mean+/−S.D. of three independent experiments; * refers to statistical significance (p<0.05); ** refers to statistical significance (p<0.01) compared with each nonsilencing control.

FIG. 7 shows the effect of TG on chemotherapeutic drugs in accordance with an embodiment of the present invention. FIGS. 7A, 7B, 7C and 7D: U-87MG cells were treated with TG (30 μM) and/or PTPI (50 μM) for 24 h. FIGS. 7A and B: treatment of cells with TG and/or PTPI was followed by treatment with VP16 (FIG. 7A, 0.01-10 μM) or Taxol (FIG. 7B, 0.005-μM) for 48 h. Cells not treated with TG or PTPI were used for control. Cells were stained with Ann and PI. Cells that stained negative for both Ann and PI were defined as viable cells. Data are mean+/−S.D. of three independent experiments; * refers to statistical significance (p<0.05); ** refers to statistical significance (p<0.01) compared with each control. FIGS. 7C and 7D: treatment of cells with TG and/or PTPI was followed by treatment with VP16 (FIG. 7C, 10 μM) or Taxol (FIG. 7D, 5 μM) for 24 h. Cells not treated with TG or PTPI were used for control. The activities of caspase-3, -8, and -9 were measured by enzyme activity assay. The specific activities on each sample were calculated according to the manufacturer's protocol. Data are mean+/−S.D. of three independent experiments; * refers to statistical significance (p<0.05); ** refers to statistical significance (p<0.01) compared with each control.

FIG. 8 illustrates potential activities of TG on TRAIL, VP16, and Taxol for facilitation of caspase cascade signaling in accordance with an embodiment of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Alleviating” specific cancers and/or their pathology includes degrading a tumor, for example, breaking down the structural integrity or connective tissue of a tumor, such that the tumor size is reduced when compared to the tumor size before treatment. “Alleviating” metastasis of cancer includes reducing the rate at which the cancer spreads to other organs.

“Beneficial results” may include, but are in no way limited to, preventing, reducing, preventing the increase of and inhibiting the proliferation or growth of cancer cells or tumors, and inducing apoptosis of cancer cells or tumor cells. Beneficial results may also refer to curing the cancer and prolonging a patient's life or life expectancy.

“Cancer” and “cancerous” refer to or describe the physiological condition in mammals that is typically characterized by unregulated cell growth. Examples of cancer include, but are not limited to, ovarian cancer, breast cancer, colon cancer, lung cancer, prostate cancer, hepatocellular cancer, gastric cancer, pancreatic cancer, cervical cancer, liver cancer, bladder cancer, cancer of the urinary tract, thyroid cancer, renal cancer, carcinoma, melanoma, head and neck cancer, and brain cancer.

“Caspase-dependent chemotherapeutic agent” as used herein refers to a chemotherapeutic agent, other than tumor necrosis factor-related apoptosis-inducing ligand (“TRAIL”), that directly or indirectly activates the caspase cascade. Examples include but are not limited to etoposide and paclitaxel.

“Conditions” and “disease conditions,” as used herein may include, but are in no way limited to any form of cancer; in particular, glioblastoma multiforme, astrocytoma, pituitary adenoma, acoustic neuroma, meningioma, oligodendrogliomas, gangliocytoma, ependymoma, medulloblastoma, medulloepithelioma, neuroblastoma, retinoblastoma, ependymoblastoma, pineocytoma, pineoblastoma, ependymal cell tumors, choroid plexus tumors, gliomatosis cerebri and astroblastoma.

“Curing” cancer includes degrading a tumor such that a tumor cannot be detected after treatment. The tumor may be reduced in size or become undetectable, for example, by atrophying from lack of blood supply, by apoptosis of the tumor cells, or by being attacked or degraded by one or more components administered according to the invention.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Pathology” of cancer includes all phenomena that compromise the well-being of the patient. This includes, without limitation, resistance to cytotoxic agents (e.g., chemotherapeutic agents), abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

“Prevention” as used herein refers to keeping a condition or disease condition from occurring even if the prevention is ultimately unsuccessful.

“Therapeutically effective amount” as used herein refers to that amount which is capable of achieving beneficial results in a patient with cancer. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, lower or reverse the chemoresistance of cancer cells or tumors or to prevent, lower or reverse the pathology of cancer cells or tumors even if the treatment is ultimately unsuccessful. Those in need of treatment include those already exhibiting chemoresistance, those prone to having chemoresistance, those in whom the chemoresistance is to be prevented, those already exhibiting the condition or disease condition, those prone to having the condition or disease condition, those in whom the condition or disease condition is to be prevented.

“Tumor,” as used herein refers to all neoplastic cell growth and proliferation, whether malignant or benign, and all pre-cancerous and cancerous cells and tissues.

“Therapeutic agent” as used herein refers to agents with the capability to prevent, inhibit, reduce, stop and/or reverse the pathology of cancer cells or tumor or the chemoresistance of cancer cells or tumors.

The present invention is directed to compositions and methods for the treatment of cancer in patients. While not wishing to be bound by any theory, the inventor has proposed a novel mechanism of proapoptotic effect induced by a pharmacological PPARγ agonist in human glioma cells (Akasaki et al., A Peroxisome Proliferator-activated Receptor-γ (PPARγ) Agonist, Troglitazone, Facilitates Caspase-8 and -9 Activities by Increasing the Enzymatic Activity of Protein Tyrosine Phosphatase-1B on Human Glioma Cells, J. Biol. Chem., 281(10), pp. 6165-6174.). The inventor demonstrated that PPARγ agonist, troglitazone (“TG” or “TGZ”), facilitates caspase-8 and -9 actions by down-regulating FLIP and Bcl-2 in human glioma cells. Troglitazone induces inactivation of signal transducer and activation of transcription-S (STAT3) and enhances the cytotoxic effects of tumor necrosis factor-related apoptosis inducing ligand (“TRAIL”) and other caspase-dependent chemotherapeutic drugs. This effect is exhibited through a PPARγ-independent mechanism, in which protein-tyrosine phosphatase 1B (PTP1B) plays a critical role in the down-regulation of activated STAT3, as well as FLIP and Bcl-2 (Akasaki et al., A Peroxisome Proliferator-activated Receptor-γ (PPARγ) Agonist, Troglitazone, Facilitates Caspase-8 and -9 Activities by Increasing the Enzymatic Activity of Protein Tyrosine Phosphatase-1B on Human Glioma Cells, J. Biol. Chem., 281(10), pp. 6165-6174.).

In particular, troglitazone induced Ser-392 phosphorylation of p53 via a PPARγ-dependent pathway and up-regulation of Bax in a p53 wild-type glioma. When given with TRAIL or caspase-dependent chemotherapeutic agents, such as etoposide and paclitaxel, troglitazone exhibited an enhancing effect on apoptosis by facilitating caspase-8/9 activities. A PPARγ antagonist, GW9662, did not block this effect, although a PTP inhibitor abrogated it. Knockdown of STAT3 by STAT3-small interfering RNA negated the inhibitory effect of PTP inhibitor on troglitazone, indicating that troglitazone uses a STAT3 inactivation mechanism that makes caspase-8/9 activities susceptible to cytotoxic agents in glioma cells and that PTP1B plays a critical role in the down-regulation of activated STAT3, as well as FLIP and Bcl-2.

Thus, the inventor has found that a combination therapy comprising a PPARγ agonist and caspase-dependent chemotherapeutic agent induces apoptosis of cancer cells, wherein the PPARγ agonist enhances the apoptotic effects of the caspase-dependent chemotherapeutic agent. Examples of caspase-dependent chemotherapeutic agents include, but are not limited to etoposide (e.g., VP-16), paclitaxel (e.g., Taxol), temozolomide, BCNU, adriamycin, cpt-11, 5-fluorouracil, oxaliplatin, pemetrexed, and gefitinib. Particularly, a combination therapy comprising both a caspase-dependent chemotherapeutic agent and a PPARγ agonist acts to induce apoptosis of cancer cells. Specifically, the present invention shows that TGZ works with each of two specific caspase-dependent chemotherapeutic drugs (VP-16 and Taxol) to increase the cytotoxic effects of these drugs in glioma cells.

In various embodiments, a treatment for disease conditions, such as cancer, includes compositions comprising at least one PPARγ agonist and one caspase-dependent chemotherapeutic agent. The compositions of the present invention may be administered to a mammal to alleviate, and potentially cure, a host of disease conditions; particularly cancer, and more particularly, cancers of the brain, such as GBM.

The caspase-dependent chemotherapeutic agents used in connection with the present invention may be selected from any caspase-dependent chemotherapeutic agent, as will be readily appreciated by one of skill in the art. Examples of such caspase-dependent chemotherapeutic agents may include, but are in no way limited to etoposide (e.g., VP-16), paclitaxel (e.g., Taxol), temozolomide, BCNU, adriamycin, cpt-11, 5-fluorouracil, oxaliplatin, pemetrexed, or gefitinib. It will also be readily appreciated by those of skill in the art that, while a single caspase-dependent chemotherapeutic agent may be administered to treat cancer in connection with various embodiments of the present invention, a wide array of combinations of caspase-dependent chemotherapeutic agents may alternatively be administered in the treatment of cancer. Moreover, caspase-dependent chemotherapeutic agents may be administered by any suitable delivery route, such as, without limitation, oral (PO), intravenous (IV), intrathecal (IT), intraarterial, intracavitary, intramuscular (IM), intralesional or topical.

The PPARγ agonists used in connection with the present invention may be selected from any PPARγ agonists, as will be readily appreciated by one of skill in the art. Examples of such PPARγ agonists may include, but are in no way limited to, troglitazone (“TG” or “TGZ”), pioglitazone (“PGZ”), rosiglitazone (“RGZ”), and ciglitazone (“CGZ”). It will also be readily appreciated by those of skill in the art that, while a single PPARγ agonist may be administered to treat cancer in connection with various embodiments of the present invention, a wide array of combinations of PPARγ agonists may alternatively be administered in the treatment of cancer. Moreover, PPARγ agonists may be administered by any suitable delivery route, such as, without limitation, oral (PO), intravenous (IV), intrathecal (IT), intraarterial, intracavitary, intramuscular (IM), intralesional or topical.

In one embodiment, the caspase-dependent chemotherapeutic agents and PPARγ agonists used in connection with the present invention may be combined in a composition using any conventional technique, as will be readily appreciated by one of skill in the art. The administration of the caspase-dependent chemotherapeutic agents and PPARγ agonists of the invention may include, without limitation, delivery of the compounds together, delivery of each compound separately, delivery as a single dosage, delivery periodically, or delivery of the compounds separately and/or at different intervals, although other schemes of administration may be used, as will be readily appreciated by those skilled in the art. “Periodically,” as used herein includes, but is in no way limited to, any interval of time such as hourly, daily, weekly, twice weekly, and monthly as would be recognized by one skilled in the art.

The quantity of the caspase-dependent chemotherapeutic agents and PPARγ agonists of the composition appropriate for administration to a patient as a cancer therapy to effectuate the methods of the present invention and the most convenient route of such administration may be based upon a variety of factors, as may the formulation of the composition itself. Some of these factors may include, but are in no way limited to, the physical characteristics of the patient (e.g., age, weight, sex, etc.), the physical characteristics of the tumor (e.g., location, size, rate of growth, accessibility, etc.), and the extent to which other therapeutic methodologies (including chemotherapy and radiation therapy) are being implemented in connection with an overall treatment regimen. Additional administrations may be effected, depending upon the above-described and other factors, such as the severity of tumor pathology.

Any conventional pharmaceutical carrier may be used with the compositions in accordance with the present invention, and an appropriate carrier may be selected by one of skill in the art by routine techniques. For example, one may formulate the pharmaceutical carrier of the composition differently in order to account for different delivery techniques for the composition, physiological differences among patients (e.g., sex, weight, age, etc.), or different types of tumors (e.g., brain, breast, lung, etc.), among other factors. The composition administered to a mammal in accordance with the present invention may be delivered in combination with any of a variety of additional substances and compounds; for example, any suitable carrier, vehicle, additive, excipient, pharmaceutical adjunct, or other suitable product. Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling may include amount, frequency, and method of administration.

A number of other chemotherapeutic agents are known to those of skill in the art and may be used, either alone or in combination with still further caspase-dependent chemotherapeutic agents and PPARγ agonists, in connection with alternate embodiments of the present invention. Many other chemotherapeutic agents will be readily recognized by those of skill in the art and can be used in connection with the present invention without undue experimentation. Examples of chemotherapeutic agents may include, but are in no way limited to, temozolomide, procarbazine, carboplatin, vincristine, BCNU, CCNU, thalidomide, irinotecan, isotretinoin (available from Hoffman-LaRoche, Inc. under the tradename Accutane®), imatinib (available from Novartis Pharmaceuticals Corporation under the tradename Gleevec®), etoposide, cisplatin, daunorubicin, doxorubicin, methotrexate, mercaptopurine, fluorouracil, hydroxyurea, vinblastine and pacitaxel. It will also be readily appreciated by those of skill in the art that, while a single chemotherapeutic agent may be administered to treat cancer in connection with various embodiments of the present invention, a wide array of combinations of chemotherapeutic agents may alternatively be administered in the treatment of cancer. Moreover, chemotherapeutic agents may be administered by any suitable delivery route, such as, without limitation, oral (PO), intravenous (IV), intrathecal (IT), intraarterial, intracavitary, intramuscular (IM), intralesional or topical.

The compositions may be administered to a mammal (e.g., a human) by any conventional technique in accordance with various embodiments of the present invention for the treatment of a disease condition, such as cancer and/or a tumor; in particular, brain cancer and/or a brain tumor. The compositions may be delivered in an amount sufficient to alleviate or cure the disease condition and/or to achieve beneficial results. The compositions may be administered by any conventional delivery route, either alone or in combination with other chemotherapeutic agents or cancer therapy (e.g., radiation therapy). The compositions may be administered by any appropriate technique, as will be readily appreciated by those of skill in the art. By way of example and not to be interpreted as limiting, the composition and/or therapy may be administered via aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

In other embodiments, a method for treating cancer or cancerous tumors in mammals in need thereof is provided. The method comprises providing compositions capable of treating, alleviating or curing the disease condition, and administering a therapeutically effective amount of the compositions to a cancer patient to treat the disease condition. The method may include providing at least one PPARγ agonist and at least one caspase-dependent chemotherapeutic agent; and implementing a combination therapy to the recipient in a manner to treat the particular condition. The caspase-dependent chemotherapeutic agents and PPARγ agonists may have characteristics similar to the compositions described above in accordance with alternate embodiments. Alternatively, the method may include providing a PPARγ agonist and administering the PPARγ agonist with radiation therapy to enhance the apoptotic effects of the radiation therapy. In another embodiment, the method may further comprise identifying a mammal in need of treatment for cancer, particularly, a cancer that is resistant to the induction of apoptosis.

There are various reasons why one might wish to administer a composition including both caspase-dependent chemotherapeutic agents and PPARγ agonists rather than administering these compounds separately in a combination therapy. Depending on the particular caspase-dependent chemotherapeutic agents and PPARγ agonists that one uses, a composition might have superior characteristics as far as clinical efficacy, solubility, absorption, stability, toxicity and/or patient acceptability are concerned. It will be readily apparent to one of ordinary skill in the art how one can formulate a composition of any of a number of combinations of caspase-dependent chemotherapeutic agents and PPARγ agonists. There are many strategies for doing so, any one of which may be implemented by routine experimentation. For example, the pharmacokinetics of the caspase-dependent chemotherapeutic agents and PPARγ agonists may determine the administration of the compounds.

The determination of a therapeutically effective dose is well within the capability of those skilled in the art. A “therapeutically effective” dose refers to that amount of active ingredient which increases or decreases the effects of a disease condition relative to that which occurs in the absence of the therapeutically effective dose. Therapeutic efficacy and toxicity, e.g., ED50 (the dose therapeutically effective in 50% of the population) and LD50 (the dose lethal to 50% of the population), can be determined by standard pharmaceutical procedures in cell cultures or experimental animals. The dose ratio of toxic to therapeutic effects is the therapeutic index, and it can be expressed as the ratio, LD50/ED50. Furthermore, the data obtained from cell culture assays and animal studies is used in formulating a range of dosage for human use, which can be readily tended to by one of ordinary skill in the art without undue experimentation. The dosage contained in such compositions may be selected so as to be within a range of circulating concentrations that include the ED50 with little or no toxicity. The dosage varies within this range depending upon the dosage form employed, sensitivity of the patient, and the route of administration.

The appropriate dosage of the caspase-dependent chemotherapeutic agents and PPARγ agonists of the invention may depend on a variety of factors. Such factors may include, but are in no way limited to, a patient's physical characteristics (e.g., age, weight, sex), whether the compound is being used as single agent or adjuvant therapy, the type of condition being treated, the progression (i.e., pathological state) of the cancer, and other factors that may be recognized by one skilled in the art. Furthermore, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans. However, the exact dosage will be determined by the practitioner, in light of factors related to the subject that requires treatment.

In a further embodiment of the present invention, a kit is provided for the treatment of cancer in a mammal. in one embodiment, the kit may be configured for cancers of the brain; for instance, for the treatment of GBM. The kit is useful for practicing the inventive method of treating disease conditions. The kit is an assemblage of materials or components. Kits for treating disease conditions may include compositions comprising at least one caspase-dependent chemotherapeutic agent and at least one PPARγ agonist capable of treating, alleviating or curing the disease condition. Furthermore, the caspase-dependent chemotherapeutic agents and PPARγ agonists may have characteristics similar to the compositions described above in accordance with alternate embodiments.

The exact nature of the components configured in the inventive kit depends on its intended purpose. For example, some embodiments are configured for the purpose of treating the disease condition of patients with cancer. In one embodiment, the kit is specifically configured for the purpose of treating the disease condition of patients with brain cancer. In various embodiments, kits may be configured particularly for the purpose of treating mammalian subjects. In other embodiments, kits are configured for veterinary applications, diagnosing or treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat, alleviate or cure a cancer patient's disease condition. Instructions may comprise instructions to administer a therapeutically effective amount of the caspase-dependent chemotherapeutic agent and the PPARγ agonist to the mammal in need of treatment for cancer. Instructions may also comprise instructions to identify a mammal in need of treatment for a cancer that is resistant to induction of apoptosis. Optionally, the kit also contains other useful components, such as diluents, buffers, pharmaceutically acceptable carriers, syringes, catheters, applicators, pipetting or measuring tools or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well known methods, preferably to provide a sterile, contaminant-free environment. The packaging materials employed in the kit are those customarily utilized in treating cancer. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Tumor Cells

A human primary cultured glioma (MG-328) was established from the surgical specimen of a patient with newly diagnosed glioblastoma at Cedars-Sinai Medical Center after Institutional Review Board-approved consent was obtained. MG-328 and human glioma cell lines, U-87MG (American Type Culture Collection, Manassas, Va.) and LN-18 (provided by Dr. Erwin Van Meier, Emory University, GA), were maintained at 37° C. and 5% CO₂ in Dulbecco's modified Eagle's medium/F-12 with 10% heat-inactivated fetal bovine serum, 2 mM glutamate, 10 mM HEPES, 100 units/ml penicillin, and 100 μg/ml streptomycin.

### Example 2

### Reagents

Recombinant human TRAIL was obtained from Pepro-Tech (Rocky Hill, N.J.). A PPARγ agonist, troglitazone (TG), was obtained from Biomol (Plymouth Meeting, Pa.). A PPARγ antagonist, GW9662 (GW), was obtained from Cayman Chemical (Ann Arbor, Mich.). PTPI, α-bromo-4-hydroxyacetophenone, and Src homology 2-containing PTP (SHP) inhibitor (SHPI), α-bromo-4-carboxymethoxyacetophenone, were obtained from Calbiochem. Etoposide (VP16) and paclitaxel (Taxol) were obtained from Sigma. TG, GW, PTPI, SHPI, VP16, and Taxol were dissolved in 100% Me₂SO as 1000× stock solution and then diluted further in culture medium. Cells were treated with rTRAIL (17.5-300 ng/ml), TG (30 μM), GW (20 μM), PTPI (50 μM), SHPI (200 μM), VP16 (0.01-10 μM), and/or Taxol (0.005-5 μM).

### Example 3

### siRNA Design and Transfection

A double-stranded siRNA oligonucleotide against STAT3 was designed as follows and as described previously (Konnikova et al. (2003) BMC Cancer 10.1186/1471-2407-3-23.): 5′-AAC AUC UGC CUA GAU CGG CUA dTdT-3′ (SEQ ID NO: 1); 3′-dTdT GUA GAC GGA UCU AGG CGA U-5′ (SEQ ID NO: 2) (STAT3-siRNA; Dharmacon RNA Technologies, Lafayette, Colo.). siRNA for nonsilencing control (nonsilencing siRNA) is an irrelevant siRNA with random nucleotides and no known specificity. Sequences were synthesized and annealed by the manufacturer (Qiagen, Valencia, Calif.) as described previously (Liu et al. (2004) Eur. J. Immunol. 34, 1680-1687.). STAT3-siRNA (50-600 pmol) or nonsilencing siRNA (600 pmol) was transfected to glioma cells (1×10⁶ cells) with Oligofectamine reagent (Invitrogen) according to the manufacturer's protocol, and the cells were used for experiments 24 h after transfection.

### Example 4

### RNA Isolation and cDNA Synthesis

Total RNA was extracted from glioma cells (1×10⁶ cells) using the RNA4PCR kit (Ambion, Austin, Tex.) according to the manufacturer's protocol. For cDNA synthesis, 1 μg of total RNA was reverse-transcribed into cDNA using oligo(dT) primer and iScript™ cDNA synthesis kit reverse transcriptase. cDNA was stored at −20° C. for PCR.

### Example 5

### Real Time Quantitative RT-PCR

Gene expression was quantified by real time quantitative reverse transcription-PCR using QuantiTect SYBR Green dye (Qiagen, Valencia, Calif.). DNA amplification was performed using an Icycler (Bio-Rad), and the binding of the fluorescence dye SYBR Green I to double-stranded DNA was measured. The PCRs were set up in microtubes at a volume of 25 μl. Oligonucleotide primers were designed as follows: STAT3 forward, 5′-GCC AGA GAG CCA GGA GCA-3′ (SEQ ID NO: 3), STAT3 reverse, 5′-ACA CAG ATA AAC TTG GTC TTC AGG TAT G-3′ (SEQ ID NO: 4); and β-actin forward, 5′-TTC TAC MT GAG CTG CGT GTG-3′ (SEQ ID NO: 5), β-actin reverse, 5′-GGG GTG TTG MG GTC TCA AA-3′ (SEQ ID NO: 6). The reaction components were 2.0 μg of cDNA synthesized as above, 12.5 μl of 2× QuantiTect SYBR Green PCR Master Mix (Qiagen, Valencia, Calif.), and 0.4 μM each pair of oligonucleotide primers. The program was as follows: initial activation for 15 min at 95° C., 50 cycles consisting of melting for 30 s at 95° C., annealing for 25 s at 60° C., and extension for 30 s at 72° C. After cycling, relative quantification of target gene mRNA against an internal control, β-actin, was possible by the following a ΔC_{T} method, and an amplification plot of fluorescence signal versus cycle number was drawn. The difference (ΔC_{T}) between the mean values in duplicated samples of the target gene and those of β-actin was calculated using Microsoft Excel, and the relative quantification value was expressed as 2^{-CΔ}^{_{T}}. The relative expression of each sample in the figure was normalized by “no treatment” expression.

### Example 6

### Antibodies

Rabbit polyclonal Abs against DR4, DR5, and PPARγ were obtained from Cayman Chemical. Mouse monoclonal Abs against STAT3, phosphotyrosine 705 STAT3 (pY705-STAT3), which is an activated form of STAT3, Bax, and Bcl-2 were obtained from Pharmingen. Rabbit polyclonal Abs against p53 and phosphor-Ser-392-p53 (pS392-p53) were obtained from Cell Signaling Technology (Beverly, Mass.). Mouse monoclonal Abs against PTP1B (PTPase1B; AE4-2J) and Src homology 2-containing PTP-1 (SHP1; 1SH01) and rabbit polyclonal Ab against FLIP were obtained from Calbiochem. Mouse monoclonal Ab of β-tubulin was obtained from Sigma. Horseradish peroxidase-linked Abs of sheep anti-mouse IgG and donkey anti-rabbit IgG were obtained from Amersham Biosciences.

### Example 7

### Western Blot

Samples were extracted with buffer containing 1% Triton X-100, 150 mM NaCl, 50 mM Tris (pH 7.5), and 1 mM phenylmethylsulfonyl fluoride (Roche Applied Science) and were subjected to SDS-PAGE with 30 μg of general protein loading into each lane on a 10% polyacrylamide gel. Electrophoretic transfer to nitrocellulose membranes (Amersham Biosciences) was followed by immunoblotting. The signal was detected by using an ECL detection system (Amersham Biosciences).

### Example 8

### Apoptosis Assay and Cell Viability Assay

Treatment of cells with TG, GW, PTPI, and/or SHPI for 24 h was followed by treatment with rTRAIL, VP16 or Taxol, and all the cells, including cells that had not adhered, were harvested after 24 h (48 h). An annexin V-fluorescein isothiocyanate apoptosis detection kit I (Pharmingen) was used for viability assay of the cells. Cells were stained with annexin V-fluorescein isothiocyanate (Ann) and propidium iodide (PI) according to the manufacturer's protocol and were analyzed by FACS (Pharmingen). Cells that stained negative for both Ann and PI were defined as viable cells for viability assay.

### Example 9

### Caspase Activity Assay

Activity of caspase-3, -8, and -9 was measured using caspase-3/CPP32, FLICE/caspase-8, and APOPCYTO/caspase-9 calorimetric assay kits, respectively, from Medical and Biological Laboratories Co. (Nagoya, Japan). Briefly, the treatment of cells with TG, GW, and/or PTPI for 24 h was followed by treatment with rTRAIL, VP 16 or Taxol. After 2 h (24 h) of treatment, all the cells were harvested, and samples were extracted with Cell Lysis Buffer. Concurrently, a sample for a negative control was extracted from the cells without apoptosis induction. A standard curve using the absorbance of p-nitroanilide standards was constructed, and then the specific activities on each sample were calculated according to the manufacturer's protocol.

### Example 10

### Statistics

Student's t test was used for statistical comparison of results.

### Example 11

### The Role of STAT3 in Resistance to Trail-Induced Apoptosis

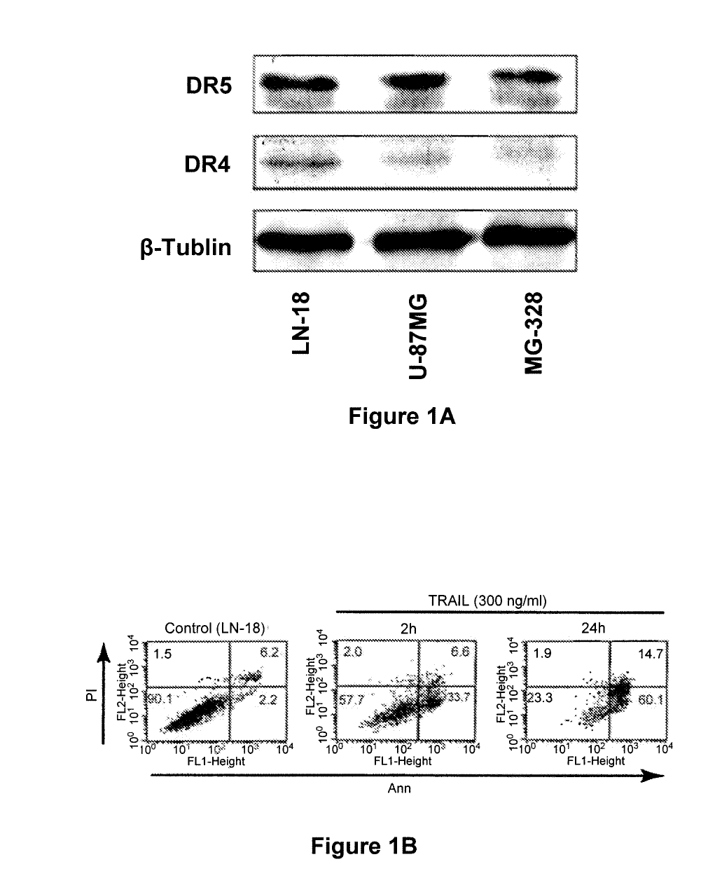

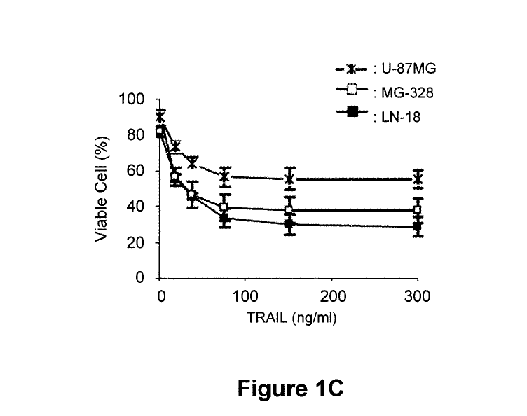

To examine the mechanism that causes resistance to TRAIL, glioma cell lines were used that are partially resistant to TRAIL despite their expression of DR4 and/or DR5. Western blot was used to detect the expression levels of DR4 and DR5 in LN-18, U-87MG, and MG-328 (FIG. 1A). The cytotoxic activity of TRAIL (18.75-300 ng/ml) was examined in each glioma (FIGS. 1, B and C). As shown in FIG. 1B, which is a representative result of FACS analysis using an annexin-V apoptosis detection kit on LN-18, treatment with TRAIL (300 ng/ml) increased the percentage of Ann(+) PI(−) cells, which indicates early stage of apoptosis, after 2 h of treatment (33.7%) and 24 h of treatment (60.1%) compared with no treatment control (2.2%). Treatment with 300 ng/ml of TRAIL induced 40-60% cell death after 24 h of treatment (FIG. 1C), although the viable cells maintained a proliferative ability after 72 h of cell culture with 300 ng/ml TRAIL (not shown). These findings indicate that these gliomas can acquire a resistance to TRAIL despite their expression of functionally intact DR4/5.

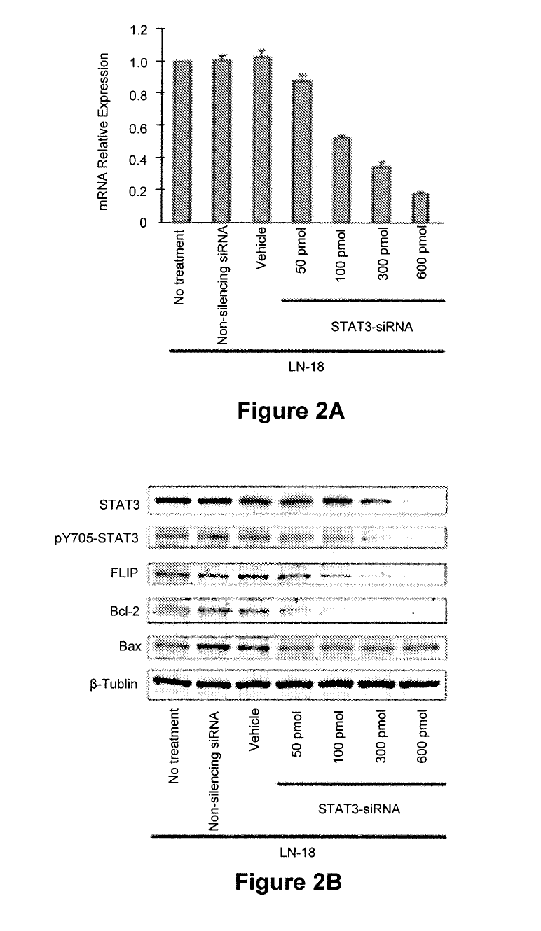

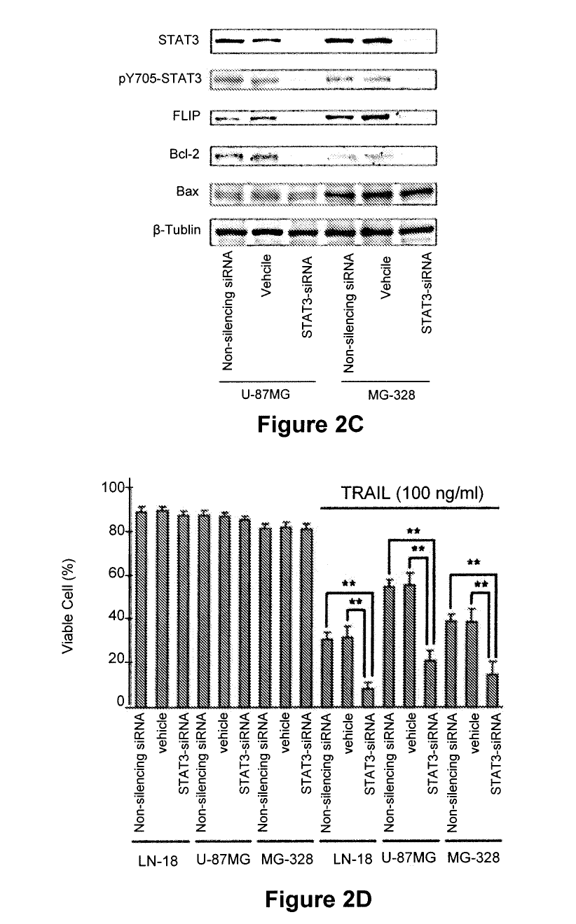

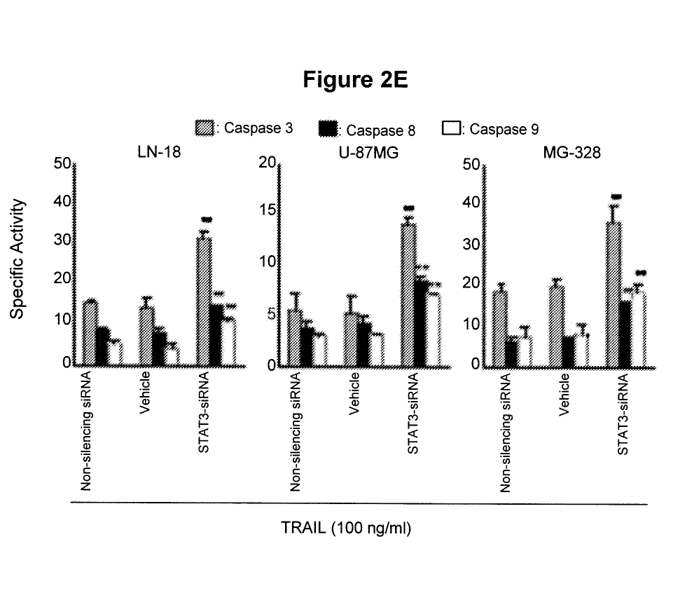

After this observation, a determination was sought whether STAT3 contributes to the resistance of gliomas to TRAIL by using STAT3-specific small interference RNA (STAT3-si RNA) (Konnikova et al. (2003) BMC Cancer 10.1186/1471-2407-3-23.). The specificity of STAT3 inhibition in LN-18 after transfection with STAT3-siRNA was investigated by real time quantitative PCR and Western blot analysis (FIGS. 2, A and B). Although neither nonsilencing control nor vehicle control had any significant change in expressions of STAT3 mRNA or STAT3 protein compared with the no treatment control, STAT3-siRNA transfection in glioma cells decreased both STAT3 mRNA (FIG. 2A) and STAT3 protein (FIG. 2B) expressions in a dose-dependent manner. These findings indicate that the STAT3-siRNA used in this study exhibited a STAT3-specific silencing effect. As shown in FIGS. 2, B and C, Western blot analysis detected high levels of STAT3, pY705-STAT3, FLIP, and Bcl-2 in the no treatment control, nonsilencing control, and vehicle control. The pY705-STAT3 was down-regulated concomitant with the decrease in STAT3 in STAT3-siRNA-transfected glioma cells (FIGS. 2, B and C). In addition, FLIP and Bcl-2 expression levels decreased in the STAT3-siRNA groups (FIGS. 2, B and C), suggesting that knockdown of STAT3 induces down-regulation of FLIP and Bcl-2 in glioma cells by inhibiting the transcriptional activity of STAT3. Bax expression levels, which is a pro-apoptotic protein induced by p53, were not altered by knockdown of STAT3 (FIGS. 2, B and C). To confirm further the regulatory function of STAT3 on the DR-signaling pathway, the nonsilencing control, vehicle control, and STAT3-siRNA-transfected cells were treated with 100 ng/ml TRAIL, and then cell viability (FIG. 20) and caspase activity assays (FIG. 2E) were performed. Transfection of STAT3-siRNA to glioma cells did not induce their apoptotic death, but it significantly enhanced the cytotoxic effect of TRAIL (FIG. 2D). The specific activities of caspases-3, -8, and -9 were also significantly increased in the siRNA-transfected group (FIG. 2E). In the assessment of the 72-h cell culture, TRAIL (100 ng/ml) killed more than 95% of the cells in the siRNA-transfected group of each glioma (not shown). These results indicate that STAT3 plays a critical role in the resistance of gliomas to TRAIL. Down-regulation of FLIP and Bcl-2 in the STAT3-siRNA-transfected group may have a particular relevance to the facilitation of the activity of caspase-3, -8, and -9.

### Example 12

### Troglitazone Activities Via PPARγ-Dependent and -Independent Pathways

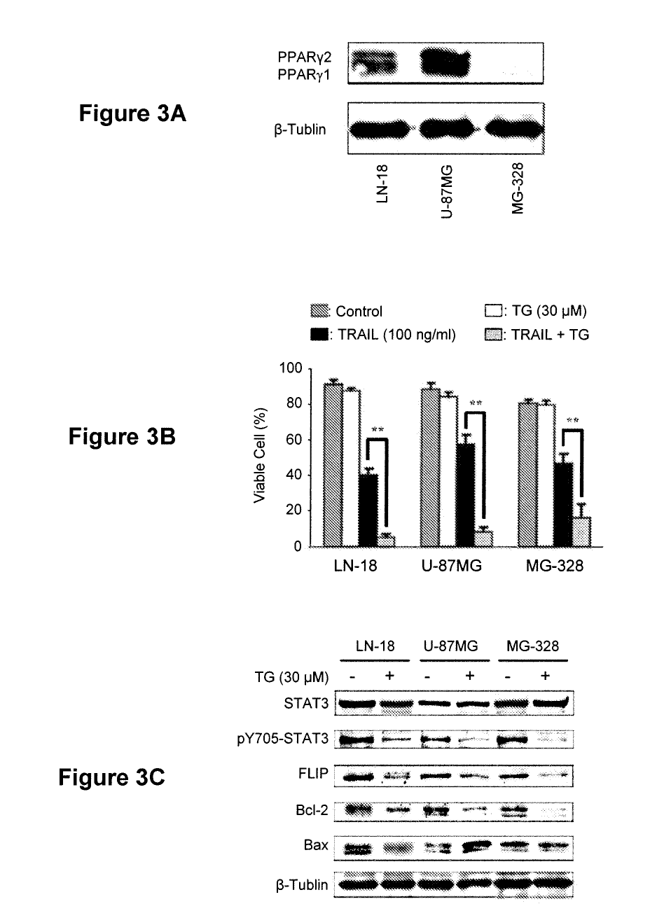

A determination was sought on whether a PPARγ agonist would enhance TRAIL-induced apoptosis. Using a Western blot analysis, PPARγ expression was observed in LN-18 and U-87 but not in MG-328 (FIG. 3A). Treatment over 48 h with TG, which is a pharmacological PPARγ agonist, did not induce apoptotic death on glioma cells but did significantly enhance the cytotoxic activity of TRAIL not only in LN-18 and U-87MG but also in MG-328 (FIG. 3B). These results suggest the possibility that the effect of TG on TRAIL is controlled by a PPARγ-independent mechanism. Although TG had no effect on STAT3 expression levels, it induced down-regulation of pY705-STAT3 (FIG. 3C). In addition, TG diminished FLIP and Bcl-2 protein expression in these cells (FIG. 3C), which was probably caused by down-regulation of pY705-STAT3. TG treatment in U-87MG, of which p53 is a wild type (Van Meir et al. (1994) Cancer Res. 54, 649-652.), increased Bax expression levels, although Bax levels were not altered in LN-18, which is a p53 mutant cell line (20), or in MG-328 (FIG. 3C). Although the sequence of p53 in MG-328 was not confirmed, this result may indicate that TGup-regulates a transcriptional activity of p53 via a PPARγ-dependent pathway, in which wild-type p53 should induce up-regulation of Bax.

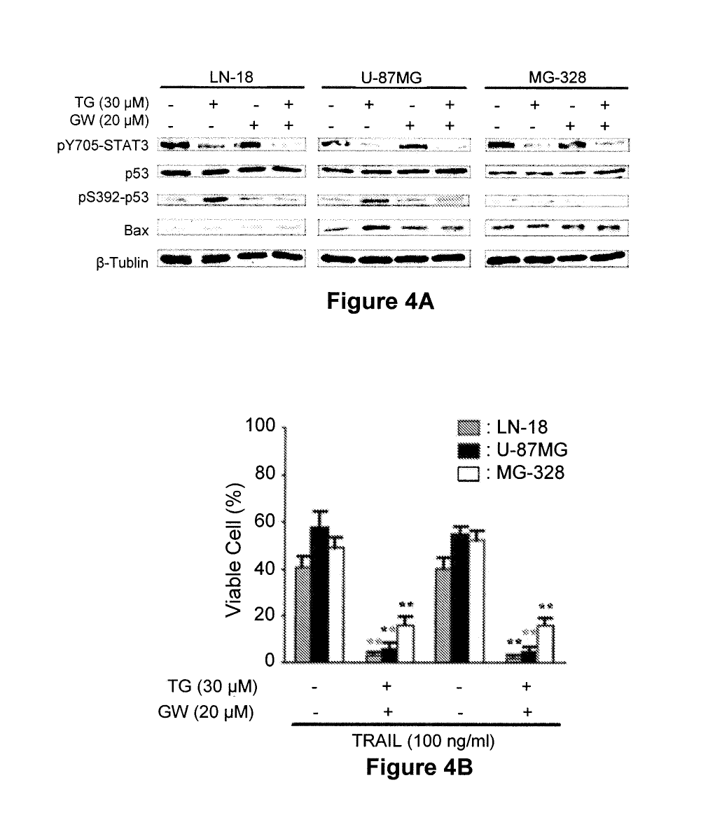

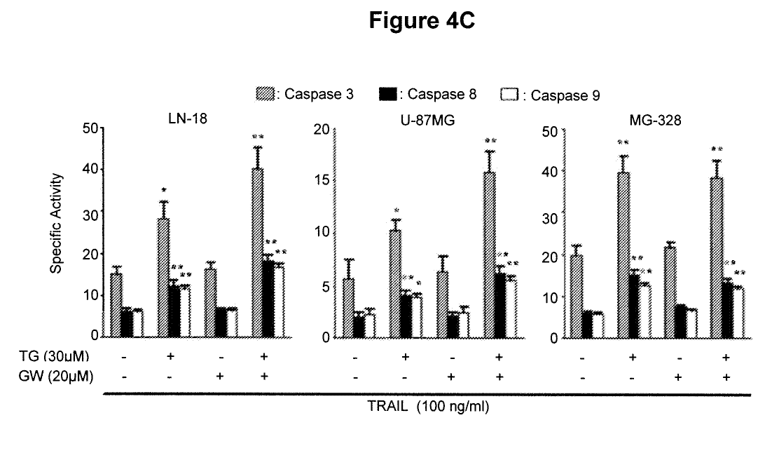

PPARγ antagonist, GW9662 (GW), was used to clarify the role of PPARγ in the effect of TG on these glioma cells. Similar to the effect of TG alone, down-regulation of pY705-STAT3 was observed on the cells treated with TG and GW (FIG. 4A), indicating that the inhibitory effect of TG on pY705-STAT3 is caused via a PPARγ-independent pathway.

In a previous report, it was demonstrated that phosphorylation of p53 at Ser-392 (pS392-p53) influenced the growth suppressor function and transcriptional activation of p53 (Kohn, K. W. (1999) Mol. Biol. Cell 10, 2703-2734.). Based on this report, the expression levels of pS392-p53 were confirmed as an index of the transcriptional activity of p53. As shown in FIG. 4A, GW abrogated the up-regulation of pS392-p53 induced by TG in LN-18 and U-87MG. GW also inhibited the up-regulation of Bax found in TG-treated U-87MG. There was no significant change in the expression levels of pS392-p53 or Bax in MG-328 in either setting (FIG. 4A). These results indicate that TG induces phosphorylation of p53 at Ser-392 via a PPARγ-dependent pathway and causes up-regulation of Bax in gliomas that have a wildtype p53. Although these results suggest that TG may have the potential to control caspase cascade signaling via both PPARγ-dependent and -independent pathways, the activity of TG in TRAIL-treated glioma cells was not blocked by a PPARγ antagonist even in U-87MG, as assessed by a cell viability assay (FIG. 4B). In addition, treatment with GW did not reduce the specific activities in any caspases that were observed (FIG. 4C), indicating that the effect of TG in TRAIL treated glioma cells occurs through a PPARγ-independent mechanism. Taken together, these results indicate that inactivation of STAT3 and the subsequent down-regulation of the downstream proteins, such as FLIP and Bcl-2, by TG may have a particular relevance to the mechanism by which TG enhances the pro-apoptotic effect of TRAIL.

### Example 13

### The Significance of PTP1B Activity in the Effect of TG

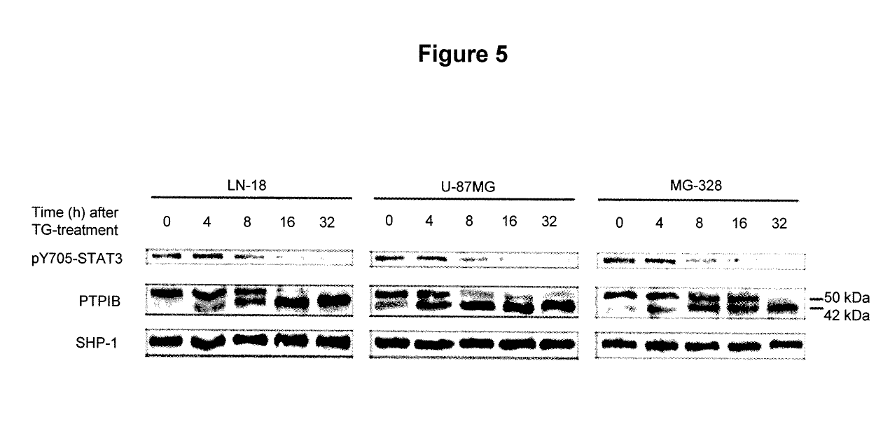

PTP1B negatively regulates tyrosine phosphorylation of JAK2 and STAT3 (Zabolotny et al. (2002) Dev. Cell 2, 489-495.; Kaszubska et al. (2002) Mol. Cell. Endocrinol. 195, 109-118.). In addition, SHP-1 negatively regulates STAT3 activity by facilitating tyrosine dephosphorylation of the upstream JAK2 (Bousquet et al. (1999) J. Clin. Investig. 104, 1277-1285.). Based on these findings, it was hypothesized that the inhibitory effect of TG on pY705-STAT3 may be caused by activation of these PTP proteins. To confirm the relationship between pY705-STAT3 and PTPs, protein samples extracted at different time points after treatment with TG were subjected to Western blot in which pY705-STAT3, PTP1B, and SHP-1 expression levels were analyzed (FIG. 5). In this assay, up-regulation of the activated form of PTP1B (42 kDa), which is induced by a proteolysis of 50-kDa PTP1B (Frangioni et al. (1992) Cell 68, 545-560; Frangioni et al. (1993) EMBO J. 12, 4843-4856.), was thought to start within 4 h after TG treatment, and its expression reached maximum levels in 16-32 h in all of these gliomas. On the other hand, down-regulation of pY705-STAT3 was noticeable at 8 h, and its expression continued to decrease after 8 h until 32 h. SHP-1 was constitutively and highly expressed in all of these gliomas, and TG had only a negligible effect on SHP-1 expression.

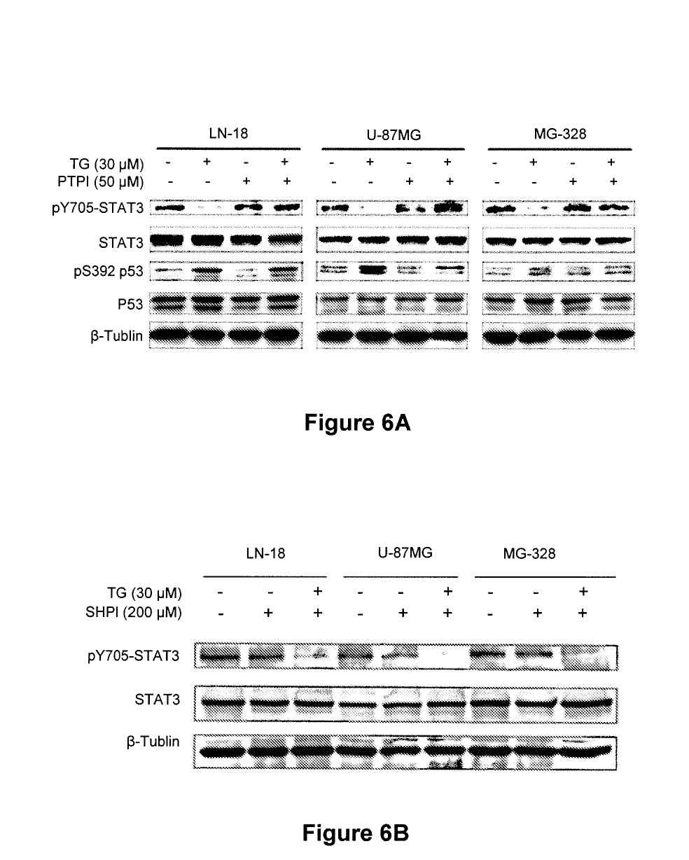

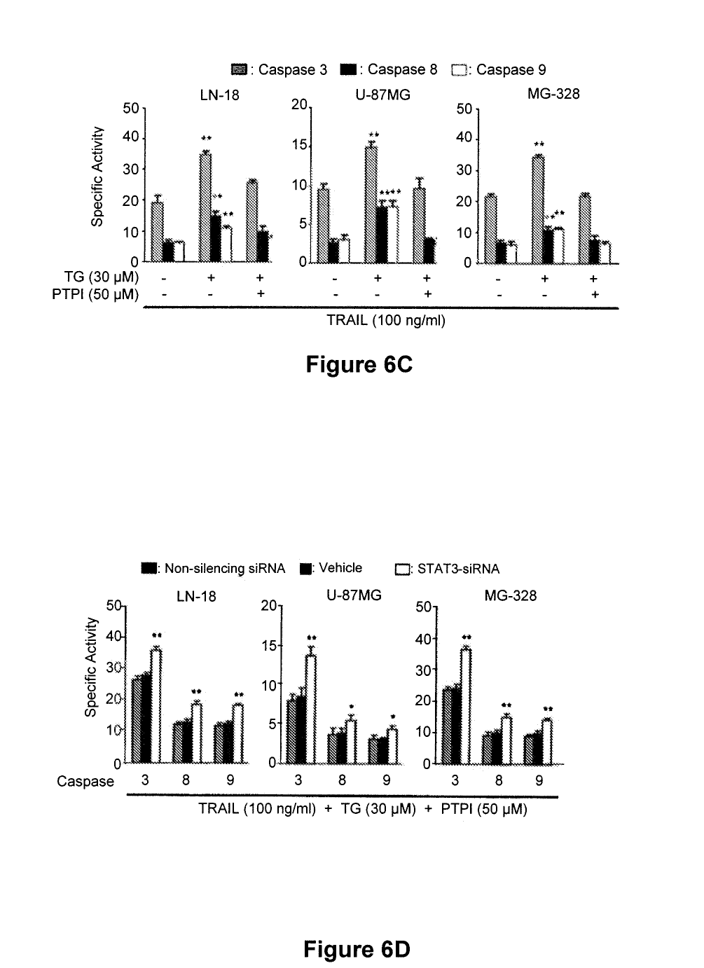

To confirm the role of PTP activity in the mechanism controlling the effect of TG on TRAIL-treated glioma cells, α-bromo-4-hydroxyacetophenone (PTPI), which is an inhibitor for both PTP1B and SHP-1, was used and α-bromo-4-carboxymethoxyacetophenone (SHPI), which is a specific inhibitor for SHP-1 (Arabaci et al. (1999) J. Am. Chem. Soc. 121, 5085-5086.). Specifically, TG treated glioma cells were co-treated with PTPI or SHPI and were then assessed by Western blot and by cell viability and caspase activity assays. In the Western blot analysis, PTPI abrogated the inhibitory effect of TG on pY705-STAT3 (FIG. 6A), whereas SHPI did not inhibit the downregulation of pY705-STAT3 induced by TG (FIG. 6B). These findings indicate that activation of PTP1B, but not SHP-1, is involved in the inhibitory effect of TG on pY705-STAT3. PTPI did not inhibit the upregulation of pS392-p53 in LN-18 and U-87MG caused by TG. Neither STAT3 nor p53 expression levels were altered in either setting (FIG. 6A). In the cell viability (not shown) and caspase activity assays (FIG. 6C), PTPI abrogated the effect of TG on TRAIL, although SHPI did not abrogate it (not shown). Thus, these results indicate that PTP1B plays a critical role in TG's effect that enhances the cytotoxic activity of TRAIL in glioma cells.

A determination was also sought whether STAT3 inactivation is a key target for PTP1B in this mechanism. To confirm this, STAT3-siRNA-transfected glioma cells were treated with TG, PTPI, and TRAIL and were then subjected to caspase activity assay. In this assessment, the inhibitory effect of PTPI on TG was negated in the STAT3-siRNA transfected glioma cells (FIG. 6D). These results indicate that activation of PTP1B by means of proteolysis of 50-kDa PTP1B and the subsequent tyrosine dephosphorylation of pY705-STAT3 is a key mechanism in this effect.

### Example 14

### The Effect of TG with Chemotherapeutic Drugs

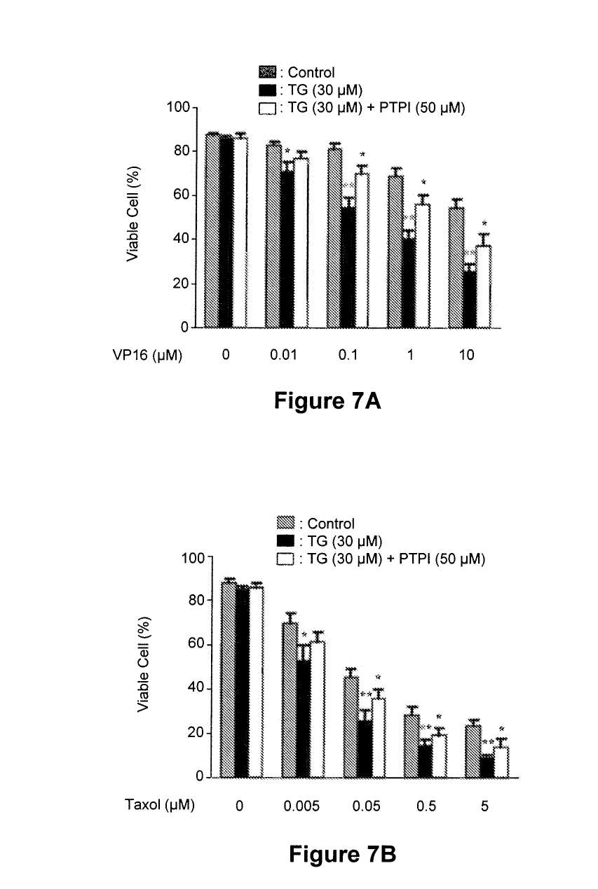

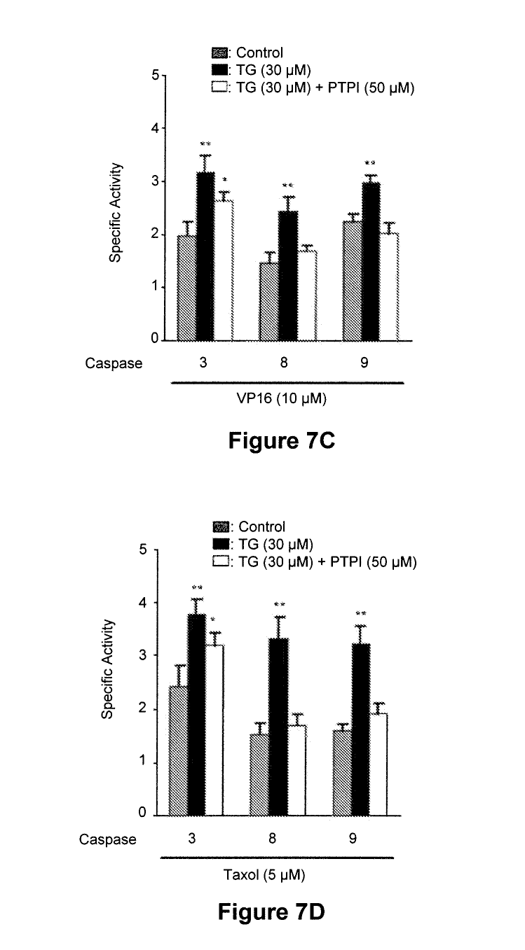

Given the already described ability of TG to facilitate caspase cascade in glioma cells by attenuating FLIP and Bcl-2 expression levels, TG may also exhibit a positive effect with chemotherapeutic drugs that activate caspase-8 and -9. Based on this hypothesis, a determination was sought whether TG enhances the cytotoxic activities of etoposide (VP16) and paclitaxel (Taxol), which are known to involve activation of caspase-2, -8, -9, and -10 during their apoptosis induction (Lin et al. (2004) J. Biol. Chem. 279, 40755-40761.; Perkins et al. (2000) Cancer Res. 60, 1645-1653.; Park et al. (2004) J. Biol. Chem. 279, 51057-51067.). To confirm this, treatment of U-87MG with TG and/or PTPI was followed by treatment with VP16 (0.01-10 μM) or Taxol (0.005-5 μM). The cells were then subjected to cell viability (FIGS. 7, A and B) and caspase activity assays (FIGS. 7, C and D). The positive activities of TG for both VP16 (FIGS. 7, A and C) and Taxol (FIGS. 7, B and D) were observed in all of the assays. Although PTPI had only a limited effect in inhibiting cytotoxic (FIGS. 7, A and B) and caspase-3 activity (FIGS. 7, C and D) in these assessments, PTPI completely abrogated the effect of TG on the activity of caspase-8 and -9 (FIGS. 7, C and D). These results indicate that TG uses a specific mechanism that makes caspase-8 and -9 activities susceptible to cytotoxic agents in glioma cells, and that PTP1B plays a critical role in the down-regulation of constitutively activated STAT3, as well as the downstream FLIP and Bcl-2 in this mechanism. Although the exact reason for the limited inhibitory effect of PTPI on cytotoxic or caspase-3 activity was not determined, these results suggest that TG may have other pro-apoptotic activities that are exhibited through a caspase-3-dependent and PTP1B-independent mechanism.

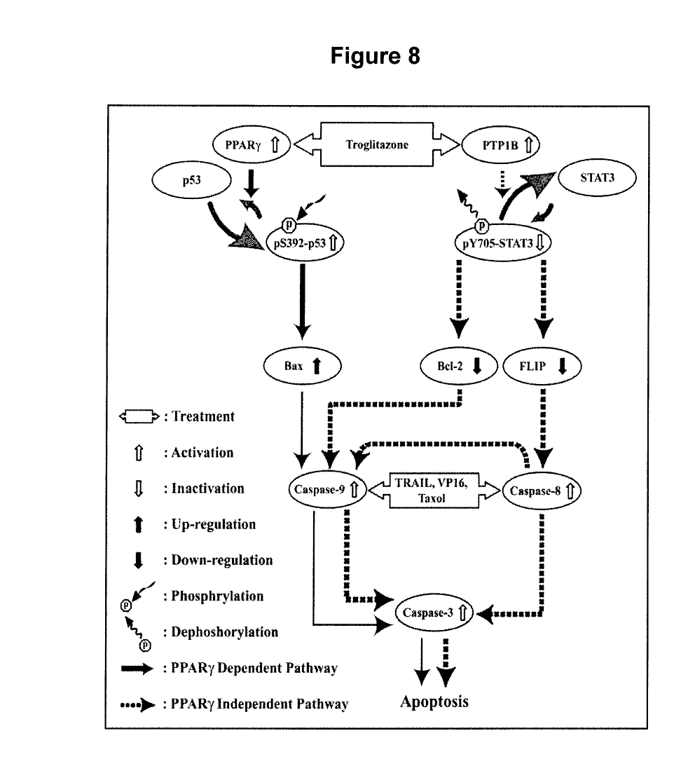

Further observation of the 5-day cell cultures in these assessments found that more than 95% of the cells in the TG and TG/PTPI treatment groups were killed by VP16 (10 μM) or Taxol (5 μM), whereas the cells in the control group maintained an ability to proliferate (not shown). Therefore, it is hypothesized that TG may be a promising drug that can abrogate the mechanism that makes malignant gliomas resistant to cytotoxic agents. Based on these results, and while not wishing to be bound by any theory, the activities of TG are illustrated in the caspase cascade (FIG. 8). Specifically, TG induces activation of PTP1B and the subsequent tyrosine dephosphorylation of constitutively activated STAT3 in glioma cells via a PPARγ-independent pathway. This event causes down-regulation of FLIP and Bcl-2 and facilitates the activities of caspase-8 and -9 when taken with caspase-dependent antineoplastic agents. TG also has the ability to induce transcriptional activation of p53 via a PPARγ-dependent pathway. In the cells with wildtype p53, this event causes up-regulation of Bax, which can facilitate caspase-9 activity, although this may be only a minor effect of the positive effect with TG. Thus, TG enhances the cytotoxic effect of caspase-dependent anti-neoplastic agents, such as TRAIL, VP16, and Taxol, by facilitating caspase cascade signaling in glioma cells. PTP1B plays a critical role in this mechanism.

### Example 15

PPARγ is a member of the nuclear hormone receptor superfamily of ligand-activated transcription factors. Interaction of PPARγ with its agonists, such as 15-deoxy-Δ^{12,14}-prostaglandin J₂ (15dPGJ2) and thiazolidenediones (TZD), exerts anti-tumor effects in a variety of cancers, indicating anti-proliferative, anti-angiogenic, and pro-differentiation effects (Koeffler, H. P. (2003) Clin. Cancer Res. 9, 1-9.). Apart from these anti-tumor activities, contradictory evidence in support of the tumor-promoting activity of PPARγ has been observed in Min mice, with a genetic predisposition to adenomatous polyposis coli (Saez et al. (1998) Nat. Med. 4, 1053-1057.). Thus, the question of whether PPARγ is a tumor suppressor gene remains controversial. Despite the oncogenic activities of PPARγ, it is now recognized that PPARγ agonists have pro-apoptotic activities, and many of them are induced through PPARγ-independent pathways (Koeffler, H. P. (2003) Clin. Cancer Res. 9, 1-9.). Therefore, PPARγ-independent mechanisms triggered by PPARγ agonists may have particular relevance to the paradoxical findings on the effect of PPARγ in cancer cells, although the mechanisms are not fully understood. Among the PPARγ-independent effects, PPARγ agonists mediate pro-apoptotic and anti-inflammatory activities by inhibiting the transcriptional activities of the STAT family, including STAT1, -3, and -5 (Nikitakis et al. (2002) Br. J. Cancer 87, 1396-1403.; Chen et al. (2003) J. Immunol. 171, 979-988.; Park et al. (2003) J. Biol. Chem. 278, 14747-14752.). Furthermore, it has been reported recently that the pro-apoptotic activity of a PPARγ agonist acting via a PPARγ-independent mechanism correlates with the inhibitory effect on FLIP and Bcl-xL/Bcl-2 functions (Kim et al. (2002) J. Biol. Chem. 277, 22320-22329.; Shiau et al. (2005) Cancer Res. 65, 1561-1569.).

It has been shown that activated STAT3 contributes to the inhibition of Fas-mediated apoptosis signaling by affecting the expression levels of FLIP and Bcl-2 (Haga et al. (2003) J. Clin. Investig. 112, 989-998.). Activation (i.e., tyrosine phosphorylation) of STAT3 is induced by signaling through JAK/STAT-associated receptors such as glycoprotein 130, growth hormone receptors, interferon receptors, and receptor tyrosine kinases (RTK), of which glycoprotein 130, exemplified by the IL-6 receptor, and RTKs, exemplified by epidermal growth factor receptor and vascular endothelial growth factor receptor, have been reported as key mediators in the inappropriate activation of STAT3 in glioma cells (Weissenberger et al. (2004) Oncogene 23, 3308-3316.; Schaefer et al. (2002) Oncogene 21, 2058-2065.; Thomas et al. (2003) Int. J. Cancer 104, 19-27.). In studies, STAT3 in glioma cells was constitutively activated, and FLIP and Bcl-2 were highly expressed. Knockdown of STAT3 by its specific siRNA facilitated caspase cascade signaling by attenuating FLIP and Bcl-2 expression. Furthermore, this effect was faithfully reproduced by a PPARγ agonist, troglitazone, which induced down-regulation of pY705-STAT3, FLIP, and Bcl-2 in glioma cells via a PPARγ-independent pathway.

It is well established that tyrosine phosphorylation is negatively regulated by PTPs, which represent a large and structurally diverse family of enzymes that rivals the protein-tyrosine kinase family, including RTKs, in structural diversity and complexity (Ostman et al. (2001) Trends Cell Biol. 11, 258-266.; Andersen et al. (2001) Mol. Cell. Biol. 21, 7117-7136.). Protein-tyrosine kinases, PTPs, and their corresponding substrates are integrated within elaborate signal transducing networks, in which PTPs can either antagonize or potentiate protein-tyrosine kinase-induced signaling events in vivo. Defective or inappropriate operation of these networks leads to aberrant tyrosine phosphorylation, which contributes to the development of many human diseases, including cancers (Hunter, T. (2000) Cell 100, 113-127.). So far, RTK activity and the effect of RTK inhibitors on malignant glioma have been noted (Li et al. (2003) Cancer Res. 63, 7443-7450.; Charest et al. (2003) Proc. Natl. Acad. Sci. U.S.A. 100, 916-921.), but the role of PTPs in gliomas is largely unknown.

In this study, expression levels of the cytoplasmic nontransmembrane PTP family, PTP1B and SHP-1, were analyzed after treatment with troglitazone. Expression levels of 42-kDa PTP1B, which is an activated form of PTP1B implicated as a negative regulator in multiple RTK signaling pathways (Ostman et al. (2001) Trends Cell Biol. 11, 258-266.; Andersen et al. (2001) Mol. Cell. Biol. 21, 7117-7136.), were extremely low in glioma cells, and troglitazone induced proteolysis of 50-kDa PTP1B to 42-kDa proteins. On the other hand, SHP-1, which negatively regulates STAT3 activity by facilitating tyrosine dephosphorylation of the upstream JAK2 on the glycoprotein 130 receptor (Bousquet et al. (1999) J. Clin. Investig. 104, 1277-1285.), was constitutively and highly expressed in all these gliomas, and troglitazone had only a negligible effect on SHP-1 expression. In the experiment using covalent inhibitors for PTP and SHP, a PTP inhibitor abrogated the effect of troglitazone on glioma cells. Furthermore, knockdown of STAT3 by STAT3-siRNA negated the inhibitory effect of a PTP inhibitor on troglitazone, indicating that troglitazone uses a STAT3 inactivation mechanism that makes caspase-8 and -9 activities susceptible to cytotoxic agents in glioma cells, and that PTP1B plays a critical role in the inhibitory effect on the signaling pathway through inappropriately activated JAK/STAT3 receptors in glioma cells.

The pharmacological mechanism by which troglitazone activates PTP1B remains unknown. PTP1B was initially described as a 37-kDa protein with tyrosine-specific protein phosphatase activity. Subsequent studies demonstrated that this purified protein represented a truncated form of a 50-kDa enzyme that is associated with the endoplasmic reticulum through interaction of its C-terminus with the cytoplasmic face (Frangioni et al. (1992) Cell 68, 545-560). Cleavage of the C-terminus of PTP1B by intracellular Ca²⁺ influx and subsequent calpain activation correlates with the appearance of a 42-kDa protein, increased PTP1B enzymatic activity, and release from the endoplasmic reticulum (Frangioni et al. (1993) EMBO J. 12, 4843-4856.; Teixeira et al. (2002) Infect. Immun. 70, 1816-1823.). A more recent study suggests that Ca²⁺ release-activated Ca²⁺ influx is inhibited by tyrosine dephosphorylation and that PTP1B plays a critical role in this dephosphorylation process (Hsu et al. (2003) Cell. Signal. 15, 1149-1156.). Palakurthi et al (Palakurthi et al. (2001) Cancer Res. 61, 6213-6218.) demonstrated that TZD induces intracellular Ca²⁺ release via a PPARγ-independent mechanism. Based on these findings, it is likely that depletion of intracellular Ca²⁺ stores by TZD treatment triggers activation of an intracellular Ca²⁺ influx in the cells exhibiting low PTP1B enzymatic activity. Therefore, Ca²⁺ release activated Ca²⁺ influx and subsequent calpain activation may be a critical mechanism by which troglitazone activates PTP1B in glioma cells via a PPARγ-independent pathway.

It was demonstrated that inactivation of constitutively activated STAT3 concomitant with PTP1B activation plays a critical role in the enhancing effect of troglitazone on the cytotoxic activities of anti-neoplastic agents in glioma cells. Although troglitazone may have the ability to facilitate caspase-3 activity and other pro-apoptotic signals through a PTP1B-independent pathway, it should be noted that troglitazone is a promising anti-neoplastic agent because of its ability to facilitate caspase-8 and -9 signaling in a PTP1B-dependent manner. These findings support the possibly enhanced effectiveness of using PPARγ agonists in clinical chemotherapy protocols that also use caspase-dependent anti-neoplastic agents such as TRAIL, etoposide, and paclitaxel for patients with malignant tumors as a means of facilitating the caspase cascade.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method of treating cancer in a mammal in need thereof comprising:
providing a caspase-dependent chemotherapeutic agent and a peroxisome proliferator-activated receptor-γ (“PPARγ”) agonist; and
administering a therapeutically effective amount of the caspase-dependent chemotherapeutic agent and a therapeutically effective amount the PPARγ agonist to the mammal in need of treatment for cancer.

2. The method of claim 1, further comprising identifying a mammal in need of treatment for a cancer that is resistant to induction of apoptosis.

3. The method of claim 1, wherein the caspase-dependent chemotherapeutic agent is selected from the group consisting of etoposide, paclitaxel, temozolomide, BCNU, adriamycin, cpt-11, 5-fluorouracil, oxaliplatin, pemetrexed, gefitinib, and combinations thereof.

4. The method of claim 3, wherein the caspase-dependent chemotherapeutic agent is etoposide.

5. The method of claim 3, wherein the caspase-dependent chemotherapeutic agent is paclitaxel.

6. The method of claim 1, wherein the PPARγ agonist is selected from the group consisting of troglitazone (“TGZ”), pioglitazone (“PGZ”), rosiglitazone (“RGZ”), ciglitazone (“CGZ”) and combinations thereof.

7. The method of claim 6, wherein the PPARγ agonist is TGZ.

8. The method of claim 1, wherein the cancer is selected from the group consisting of breast cancer, colon cancer, prostate cancer, pancreatic cancer, cervical cancer, thyroid cancer, brain cancer and combinations thereof.

9. The method of claim 1, wherein the cancer is malignant glioma.

10. The method of claim 9, wherein the malignant glioma is gliobastoma multiforme or anaplastic astrocytoma.

11. The method of claim 1, wherein the cancer is resistant to induction of apoptosis.

12. A composition for the treatment of cancer in a mammal in need thereof comprising:
a caspase-dependent chemotherapeutic agent; and
a peroxisome proliferator-activated receptor-γ (“PPARγ”) agonist.

13. The composition of claim 12, wherein the caspase-dependent chemotherapeutic agent is selected from the group consisting of etoposide, paclitaxel, temozolomide, BCNU, adriamycin, cpt-11, 5-fluorouracil, oxaliplatin, pemetrexed, gefitinib, and combinations thereof.

14. The composition of claim 13 wherein the caspase-dependent chemotherapeutic agent is etoposide.

15. The composition of claim 13, wherein the caspase-dependent chemotherapeutic agent is paclitaxel.

16. The composition of claim 12, wherein the PPARγ agonist is selected from the group consisting of troglitazone (“TGZ”), pioglitazone (“PGZ”), rosiglitazone (“RGZ”), ciglitazone (“CGZ”) and combinations thereof.

17. The composition of claim 16, wherein the PPARγ agonist is TGZ.

18. A kit for the treatment of cancer in a mammal in need thereof, comprising:
a quantity of a caspase-dependent chemotherapeutic agent;
a quantity of a peroxisome proliferator-activated receptor-γ (“PPARγ”) agonist; and
instructions to administer a therapeutically effective amount of the caspase-dependent chemotherapeutic agent and a therapeutically effective amount the PPARγ agonist to the mammal in need of treatment for cancer.

19. The kit of claim 18, further comprising instructions to identify a mammal in need of treatment for a cancer that is resistant to induction of apoptosis.

20. The kit of claim 18, wherein the caspase-dependent chemotherapeutic agent is selected from the group consisting of etoposide, paclitaxel, temozolomide, BCNU, adriamycin, cpt-11, 5-fluorouracil, oxaliplatin, pemetrexed, gefitinib, and combinations thereof.

21. The kit of claim 20, wherein the caspase-dependent chemotherapeutic agent is etoposide.

22. The kit of claim 20, wherein the caspase-dependent chemotherapeutic agent is paclitaxel.

23. The kit of claim 18, wherein the PPARγ agonist is selected from the group consisting of troglitazone (“TGZ”), pioglitazone (“PGZ”), rosiglitazone (“RGZ”), ciglitazone (“CGZ”) and combinations thereof.

24. The kit of claim 23, wherein the PPARγ agonist is TGZ.

25. The kit of claim 18, wherein the cancer to be treated is selected from the group consisting of breast cancer, colon cancer, prostate cancer, pancreatic cancer, cervical cancer, thyroid cancer, brain cancer and combinations thereof.

26. The kit of claim 18, wherein the cancer to be treated is malignant glioma.

27. The kit of claim 26, wherein the malignant glioma is gliobastoma multiforme or anaplastic astrocytoma.

28. The kit of claim 18, wherein the cancer to be treated is a cancer that is resistant to induction of apoptosis.
